(19) United States
(12) Patent Application Publication
(10) Pub. No.: US 2008/0267900 A1
(43) Pub. Date: Oct. 30, 2008

(54) Surface Treatment

(75) Inventors: Ulrich Steinbrenner, Neustadt (DE); Wolfgang Schul, Bensheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/909,526

(22) PCT Filed: Mar. 22, 2006
(86) PCT No.: PCT/EP06/60945

§ 371 Date: Nov. 2, 2007

(30) Foreign Application Priority Data
Mar. 23, 2005 (DE) 10 2005 014 033.5

(51) Int. Cl. A61L 9/01

(52) U.S. Cl. 424/76.1

(57) ABSTRACT

The present invention relates to a method of treating solid surfaces where a suspendable liquid mist provided with suitable surfactants is applied to the surface to be treated, and the liquid mist drops have a surfactant content of from 10 to 3000 ppm by weight, based on the total amount of solvent, and an average drop size (weight-average) of ≦100 μm and to the use of surfactant-containing suspendable liquid mists for deodorization, decontamination, disinfection, corrosion protection, preservation, the stripping of solid surfaces or the placing of beneficial animals and microorganisms onto these.

The present invention relates to a method of treating solid surfaces by applying a suspendable liquid mist provided with suitable surfactants to the surface to be treated, and to the use of surfactant-containing suspendable liquid mists for deodorization, decontamination, disinfection, corrosion protection, preservation, the stripping of solid surfaces or the placing of beneficial animals and microorganisms onto these.

EP 0 972 556 B1 discloses a process and methods for the adsorption of hydrophobic gas components and/or aerosols from a gas phase by means of a suspendable liquid mist enriched with surfactants, and the use of this liquid mist for the adsorption of hydrophobic gas components and/or aerosols. The binding of the gas components and/or of the aerosols takes place via the physical adsorption onto surfactants on the surface of the liquid mist drops. The subject-matter of EP 0 972 556 B1 can be used in wide areas of processing technology, production and offgas purification.

DE 100 63 010 C1 discloses a method of moistening and/or charging biomass with substances by suspendable mist which is introduced into the biomass from below in a directional stream of air. The suspendable mist passes through this biomass in the direction from bottom to top and is absorbed within the biomass. The invention according to DE 100 63 010 C1 is used in technical fields in which biomasses are present in nonaqueous form as solid or applied to a solid.

DE 100 40 015 A1 discloses a method of depositing hot gases and/or hot dusts onto colder surfactant-coated liquid drops and devices for carrying out this method and its use for cleaning waste air, particularly in the bitumen-processing industry.

A method of treating solid surfaces with a suspendable liquid mist provided with suitable surfactants is not known to date.

The object of the present invention is therefore to provide a method of treating solid surfaces with a suspendable liquid mist which is provided with suitable surfactants.

This object is achieved by a method of treating solid surfaces where a suspendable liquid mist provided with suitable surfactants is applied to the surface to be treated, where the liquid mist drops have a surfactant content of from 10 to 3000 ppm by weight, based on the total amount of solvent, and an average drop size (weight-average) of ≦100 μm.

The method according to the invention has the advantage that the very small solvent drops serve as vehicles which can apply active agents also into cracks, gaps and niches of the surface with a low vapor pressure. As a result, pores of surfaces are, for example, better reached by the active agents.

It is of particular importance for carrying out the method according to the invention that the liquid mist used is a suspendable liquid mist. Firstly, the use of a suspendable very fine mist as a carrier of the active agents leads to a considerable enlargement of the surface of the mist, and secondly to a considerable reduction in the average distance between the drops. As a result, a very rapid and uniform coating of the surface to be treated with the mist droplets and thus with the active agents is achieved. This fact in particular is of particular importance with regard to the low diffusion rate of relatively large organic, hydrophobic active agents which, if applied inhomogeneously to the surface, would only lead to a homogeneous distribution with difficulty as a result of intrinsic diffusion. These properties of a suspendable liquid mist, in combination with an adsorptive surface of the individual very fine droplets produced by means of surfactants, bring about an unexpectedly efficient wetting of the surface to be treated.

In a preferred embodiment, the treatment is a deodorization, for example the removal of unpleasant or harmful odors, decontamination, for example the removal of solid or liquid, toxic or harmful substances, disinfection, corrosion protection, for example of car, lorry or ship bodywork, preservation, for example of foods, cosmetics or body care compositions, stripping of polymer layers, for example of colors, paints and/or coatings or placing of beneficial animals and/or microorganisms, preferably beneficial microorganisms. The method according to the invention can be used to treat all natural and synthetic surfaces irrespective of their nature as regards porosity, structure or composition.

In a preferred embodiment, the surfaces are chosen from the group consisting of polymer surfaces, metallic surfaces, ceramic surfaces, porcelain surfaces, glass surfaces, wood surfaces, paint surfaces, textile surfaces, surfaces of plants, animal or people, leather and hides.

The specified surfaces may arise in the following areas: hospitals, doctors surgeries, military, nuclear power stations, laboratories, kitchens and canteens, wet rooms, baths and toilets, greenhouses, stables, zoos, smoking areas, apartments, hotel rooms, production plants, interiors of cars, composting works, refuse dumps and heaps, shells of buildings or farming and forestry.

According to one embodiment, the method according to the invention does not relate to the treatment of surfaces of biomasses. Biomasses are understood as meaning all solid masses made of biological material themselves or biological material on solid as support material. Solid material is understood by the person skilled in the art as meaning material which is insoluble in water.

The use of a suspendable liquid mist for the purposes of the invention is not only advantageous, but decisive for the desired efficiency of the method according to the invention.

The expression “suspendable mist” for the purposes of the present invention preferably refers to a drop size which brings about a fall velocity of ≦100 cm per minute, preferably ≦20 cm per minute, particularly preferably <10 cm per minute at atmospheric pressure in the motionless air or gas phase.

Such a suspendable mist arises below an average drop size (weight-average) of 100 μm diameter. An average drop size (weight-average) with a diameter of from 1 to 100 μm is a preferred drop size for carrying out the method according to the invention. In a particularly preferred embodiment the present invention relates to a method in which a suspendable mist having an average drop size (weight-average) of from 1 to 50 μm, very particularly preferably 1 to 30 μm, in particular 10 to 20 μm, is used.

Average drop sizes (weight-average) of from 10 to 20 μm diameter can be realized industrially using high-pressure nozzles. Alternatively, ultrasound nebulizers or nebulizers in which the atomization proceeds at high-speed surfaces, for example rotating discs, are possible.

The suspendability of the liquid mist increases many times over the ability to penetrate into cracks, gaps and niches of the surface to be treated.

The method according to the invention also has the advantage that, due to the low drop size, much less solvent has to be applied to the surface to be treated in order to achieve the desired effect of complete and homogeneous wetting than is known by methods from the prior art

In the method according to the invention, the suspendable liquid mist is provided with a suitable surfactant.

Surfactants are so-called amphiphilic molecules which have a hydrophobic moiety and a hydrophilic moiety in their molecular structure. As a result of this property, surfactants can form so-called micelles. These are aggregates of surfactants which can form in aqueous solutions and assume various forms (cones, rods, discs). Micelles form above a certain concentration, the so-called critical micelle concentration (CMC). In addition, amphiphilic molecules have the property of forming interfacial films between hydrophobic and hydrophilic phases and thus, for example, of having an emulsifying effect.

The liquid mist drops of the invention are characterized in that the addition of a suitable amount of a suitable surfactant to the aqueous, i.e. polar, initial charge and subsequent very fine nebulization of the aqueous surfactant solution rapidly leads to an orientation of the added surfactants to the surface of the generated very fine mist drops. The polar hydrophilic moiety of the surfactant molecule remains in the polar aqueous phase of the drop, the nonpolar hydrophobic moiety stretches from the surface of the drop out into the surrounding air.

This desired effect can be optimized through the choice of the molecular structure of the surfactants suitable for this purpose. Sterically hindered hydrophobic chains have proven particularly advantageous for this.

In the optimum state (at a suitable surfactant concentration), the aqueous polar liquid drop should have a surface which is virtually completely coated with nonpolar hydrophobic material. A suitable concentration of surfactants in the mist drops to the optimally surfactant-coated surface is dependent both on the drop size and also on the surfactant used. The concentration range of the surfactants which can be used is governed by the “water consumption” of the hydrophilic moiety or of the spatial expansion of the hydrophobic moiety of the amphiphilic surfactants.

In a preferred embodiment, surfactants chosen from cationic, nonionic, zwitterionic, anionic surfactants and mixtures of two or more of said surfactants are used.

Cationic Surfactants

Preferred cationic surfactants are chosen from the group of quaternary diester ammonium salts, quaternary tetraalkyl ammonium salts, quaternary diamido ammonium salts, amidoamine esters and imidazolium salts. Examples are quaternary diester ammoniur salts which have two C₁₁- to C₂₂-alk(en)yl-carbonyloxy(mono- to pentamethylene) radicals and two C₁- to C₃-alkyl or hydroxyalkyl radicals on the quaternary N atom and carry, as counterion, chloride, bromide, methylsulfate or sulfate, for example.

Quaternary diester ammonium salts are also in particular those which have a C₁₁- to C₂₂-alk(en)ylcarbonyloxytrimethylene radical which carries a C₁₁- to C₂₂-alk(en)ylcarbonyloxy radical on the middle carbon atom of the trimethylene group, and three C₁- to C₃-alkyl or hydroxyalkyl radicals on the quaternary N atom and carry, as counterion, chloride, bromide, methylsulfate or sulfate, for example.

Quaternary tetraalkyl ammonium salts are, in particular, those which have two C₁- to C₆-alkyl radicals and two C₈- to C₂₄-alk(en)yl radicals on the quaternary N atom and carry, as counterion, chloride, bromide, methylsulfate or sulfate, for example.

Quaternary diamido ammonium salts are in particular those which have two C₈- to C₂₄-alk(en)ylcarbonylaminoethylene radicals, a substituent chosen from hydrogen, methyl, ethyl and polyoxyethylene having up to 5 oxyethylene units and, as fourth radical, a methyl group on the quaternary N atom and carry, as counterion, chloride, bromide, methylsulfate or sulfate, for example.

Amidoamino esters are in particular tertiary amines which carry as substituents on the N atom a C₁₁- to C₂₂-alk(en)ylcarbonylamino(mono- to trimethylene) radical, a C₁₁- to C₂₂-alk(en)ylcarbonyloxy(mono- to trimethylene) radical and a methyl group.

Imidazolinium salts are in particular those which carry a C₁₄- to C₁₈-alk(en)yl radical in the 2-position of the heterocycle, a C₁₄- to C₁₈-alk(en)ylcarbonyl(oxy or amino)ethylene radical on the neutral N atom and hydrogen, methyl or ethyl on the N atom carrying the positive charge. Counterions here are, for example, chloride, bromide, methylsulfate or sulfate.

Nonionic Surfactants

Suitable nonionic surfactants are in particular alkoxylated C₆- to C₂₂-alcohols, such as fatty alcohol alkoxylates or oxo alcohol alkoxylates. These can be alkoxylated with ethylene oxide, propylene oxide and/or butylene oxide. Surfactants which can be used here are all alkoxylated alcohols to which at least two molecules of one of the abovementioned alkylene oxides have been added. Of suitability here are block polymers of ethylene oxide, propylene oxide, butylene oxide, styrene oxide, isobutylene oxide, pentene oxide or decene oxide, or addition products which comprise the specified alkylene oxides in random distribution. Per mole of alcohol, the nonionic surfactants comprise generally 2 to 50, preferably 3 to 20, mol of at least one alkylene oxide. Preferably, these comprise ethylene oxide as alkylene oxide. The alcohols preferably have 6 to 13 carbon atoms. Depending on the nature of the alkoxylation catalyst used in the preparation, the alkoxylates have a broad or narrow alkylene oxide homolog distribution.

Suitable nonionic surfactants are also alkylphenol alkoxylates, such as alkylphenol ethoxylates with C₆- to C₁₂-alkyl chains and 5 to 30 alkylene oxide units, alkyl polyglucosides having 8 to 22, preferably 10 to 18, carbon atoms in the alkyl chain and in general 1 to 20, preferably 1.1 to 5, glucoside units, N-alkylglucamides. fatty acid amide alkoxylates, fatty acid alkanolamide alkoxylates, and block copolymers of ethylene oxide, propylene oxide and/or butylene oxide.

Preferred alcohol ethoxylates have an HLB value according to W. C. Griffin. i.e. 20 times the mass fraction of ethylene oxide in the molecule, between 2 and 19, particularly preferably between 6 and 15, very particularly preferably between 8 and 14.

Preferred polyalkylene oxides and alcohol alkoxylates, e.g. EO-PO block copolymers and surfactants of the composition C₆- to C₂₂-alkyl-(EO, PO, BuO, PeO)_{y}-OH, where block and random structures are possible, have an HLB value—here calculated as 20 times the mass fraction of ethylene oxide plus 10 times the mass fraction of propylene oxide—between 2 and 19, particularly preferably between 6 and 15, very particularly preferably between 8 and 14.

Particularly preferred nonionic surfactants are hexanol ethoxylates, 2-ethylhexanol ethoxylates, 2-propylheptanol ethoxylates and isotridecyl ethoxylates.

Zwitterionic Surfactants

In the method according to the invention, zwitterionic surfactants which can be used are all surface-active substances with at least two functional groups which can ionize in aqueous solution and in so doing, depending on the conditions of the medium, impart anionic or cationic character to the surface-active compounds.

Zwitterionic surfactants which can be used according to the invention include betaines, amine oxides, alkylamidoalkylamines, alkyl-substituted amino acids, acetylated amino acids and surfactants of natural origin, such as lecithins or saponins.

Betaines

Suitable betaines are the alkylbetaines, the alkylamidobetaines, the imidazoliniumbetaines, the sulfobetaines and the phosphobetaines and preferably satisfy formula (I),

R¹—[CO—X-(CH₂)ₙ]ₓ—N⁺(R²)(R³)−(CH₂)ₘ—[CH(OH)—CH₂]_{y}—Y⁻ (I),

in which

R¹ is a saturated or unsaturated C₆-₂₂-alkyl radical, preferably C₈-₁₈-alkyl radical, in particular a saturated C₁₀-₁₆-alkyl radical, for example a saturated C₁₂-₁₄-alkyl radical,

X is NH, NR⁴ with the C₁₋₄-alkyl radical R⁴, O or S,

n is a number from 1 to 10, preferably 2 to 5, in particular 3,

x is 0 or 1, preferably 1,

R², R³, independently of one another, are a C₁₋₄-alkyl radical, optionally hydroxy-substituted, such as, for example, a hydroxy ethyl radical, in particular a methyl radical,

m is a number from 1 to 4, in particular 1, 2 or 3,

y is 0 or 1 and

Y is COO, SO₃, OPO(OR⁵)O or P(O)(OR⁵)O, where R⁵ is a hydrogen atom or a C₁-C₄-alkyl radical.

The alkyl- and alkylamidobetaines, betaines of the formula (I) with a carboxylate group (Y═COO—), are also called carbobetaines.

Further zwitterionic surfactants are the alkylbetaines of the formula (II), the alkylaminobetaines of the formula (III), the sulfobetaines of the formula (IV) and the amidosulfobetaines of the formula (V),

R¹—N⁺(CH₃)₂—CH₂COO⁻ (II)

R¹—CO—NH—(CH₂)₃—N⁺(CH₃)₂—CH₂COO⁻ (III)

R¹—N⁺(CH₃)₂—CH₂CH(OH)CH₂SO₃⁻ (IV)

R¹—CO—NH—(CH₂)₃—N⁺(CH₃)₂CH₂CH(OH)CH₂SO₃⁻ (V)

in which R¹ has the same meaning as in formula (I).

Examples of suitable betaines and sulfobetaines are the following compounds: Almondamidopropyl Betaine, Apricotamidopropyl Betaine, Avocadamidopropyl Betaine, Babassuamidopropyl Betaine, Behanamidopropyl Betaine, Behenyl Betaine, Betaine, Canolamidopropyl Betaine, Capryl/Capramidopropyl Betaine, Carnitine, Cetyl Betaine, Cocamidoethyl Betaine, Cocamidopropyl Betaine, Cocaridopropyl Hydroxysultaine, Coco-Betaine, Coco-Hydroxysultaine, Coco/Oleamidopropyl Betaine, Coco-Sultaine, Decyl Betaine, Dihydroxyethyl Oleyl Glycinate, Dihydroxyethyl Soy Glycinate, Dihydroxyethyl Stearyl Glycinate, Dihydroxyethyl Tallow Glycinate, Dimethicone Propyl PB-Betaine, Erucamidopropyl Hydroxysultaine, Hydrogenated Tallow Betaine, Isostearamidopropyl Betaine, Lauramidopropyl Betaine, Lauryl Betaine, Lauryl Hydroxysultaine, Lauryl Sultaine, Milkamidopropyl Betaine, Minkamidopropyl Betaine, Myristamidopropyl Betaine, Myristyl Betaine, Oleamidopropyl Betaine, Oleamidopropyl Hydroxysultaine, Oleyl Betaine, Olivamidopropyl Betaine, Palmamidopropyl Betaine, Palmitamidopropyl Betaine, Palmitoyl Carnitine, Palm Kernelamidopropyl Betaine, Polytetrafluoroethylene Acetoxypropyl Betaine, Ricinoleamidopropyl Betaine, Sesamidopropyl Betaine, Soyamidopropyl Betaine, Stearamidopropyl Betaine, Stearyl Betaine, Tallowamidopropyl Betaine, Tallowamidopropyl Hydroxysultaine, Tallow Betaine, Tallow Dihydroxyethyl Betaine, Undecylenamidopropyl Betaine and Wheat Gerrn idopropyl Betaine.

Amine Oxides

Amine oxides suitable according to the invention as arphoteric surfactants include alkylamine oxides, in particular alkyldimethylamine oxides, alkylamidoamine oxides and alkoxyalkylamine oxides. Preferred amine oxides satisfy formulae (VI) and (VII),

R⁶R⁷R⁸N⁺O⁻ (VI)

R⁶—[CO—NH—(CH₂)_{w}]_{z}—N⁺(R⁷)(R⁸)—O⁻ (VII)

in which R⁶ is a saturated or unsaturated C₆-₂₂-alkyl radical, preferably C₈₋₁₈-alkyl radical, in particular a saturated C₁₀₋₁₆-alkyl radical, for example a saturated C₁₂₋₁₅-alkyl radical which is bonded to the nitrogen atom N in the alkylamidoamine oxides via a carbonylamidoalkylene group —CO—NH—(CH₂)₂— and in the alkoxyalkylamine oxides via an oxaalkylene group —O—(CH₂)_{z}, where z is in each case a number from 1 to 10, preferably 2 to 5, in particular 3, R⁷, R⁸ independently of one another, are a C₁₋₄-alkyl radical, optionally hydroxy-substituted, such as, for example, a hydroxyethyl radical, in particular a methyl radical.

Examples of suitable amine oxides are the following compounds: Almondamidpropylamine Oxide, Babassuaamidopropylamine Oxide, Behenamine Oxide, Cocamidopropylamine Oxide, Cocamine Oxide, Coco-Morpholine Oxide, Decylamine Oxide, Decyltetradecylamine Oxide. Diaminopyrimidine Oxide, Dihydroxyethyl-C₈₋₁₀-alkoxypropylamine Oxide, Dihydroxyethyl-C₉₋₁₁-alkoxypropylamine Oxide, Dihydroxyethyl-C₁₂₋₁₅-alkoxypropylamine Oxide, Dihydroxyethyl Lauramine Oxide, Dihydroxyethyl Stearamine Oxide, Dihydroxyethyl Tallowamine Oxide, Hydrogenated Palm Kernel Amine Oxide, Hydrogenated Tallowamine Oxide, Hydroxyethyl Hydroxypropyl-C₁₂₋₁₅-alkoxypropylamine Oxide, Isostearamidopropylamine Oxide, Isostearamidopropyl Morpholine Oxide, Lauramidopropylamine Oxide, Lauramine Oxide, Methyl Morpholine Oxide, Milkamidopropyl Amine Oxide, Minkamidopropylamine Oxide, Myristamidopropylamine Oxide, Myristamine Oxide, Myristyl/Cetyl Amine Oxide, Oleamiopropylamine Oxide, Oleamine Oxide, Olivamidopropylamine Oxide, Palmitamidopropylamine Oxide, Palnunitamine Oxide, PEG-3 Lauramine Oxide, Potassium Dihydroxyethyl Cocamine Oxide Phosphate, Potassium Triphosphonomethylamine Oxide, Sesamidopropylamine Oxide, Soyamidopropylamine Oxide, Stearamidopropylamine Oxide, Stearamine Oxide, Tallowamidopropylamine Oxide, Tallowamine Oxide, Undecyleneamidopropylamine Oxide, Wheat Gerrn idopropylamine Oxide, Cocoyldimethylamine oxide, Lauryldimethylamine oxide, Decyldimethylamine oxide and Myristyldimethylamine oxide.

Alkylamidoalkylarines

The alkylamidoalkylamines are amphoteric surfactants of the formula (VIII),

R⁹—CO—(NR¹⁰—(CH₂)ᵢ—N(R¹¹)—(CH₂CH₂O)ⱼ—(CH₂)ₖ—[CH(OH)]ₗ—CH₂-Z-OM² (VII)

in which R⁹ is a saturated or unsaturated C₆₋₂₂-alkyl radical, preferably C₈₋₁₈-alkyl radical, in particular a saturated C₁₀₋₁₆-alkyl radical, for example a saturated C₁₂₋₁₃-alkyl radical.

R¹⁰ is a hydrogen atom H or a C₁₋₄-alkyl radical, preferably H,

i is a number from 1 to 10, preferably 2 to 5, in particular 2 or 3,

R¹¹ is hydrogen or CH₂COOM² (for M² see below)

j is a number from 1 to 4, preferably 1 or 2, in particular 1,

k is a number from 0 to 4, preferably 0 or 1,

l is 0 or 1,

Z is CO, SO₂, OPO(OR¹²) or P(O)(OR¹²), where R¹² is a C₁₋₄-alkyl radical or is M² (see below), and

M² is a hydrogen atom, an alkali metal, an alkaline earth metal or a protonated alkanolarine, e.g. protonated mono-, di- or triethanolamine. Preferred representatives satisfy the formulae (IX) to (XII),

R⁹—CO—NH—(CH₂)₂—N(R¹¹)—CH₂CH₂O—CH₂—COOM² (IX)

R⁹—CO—NH—(CH₂)₂—N(R¹¹)—CH₂CH₂O—CH₂CH₂—COOM² (X)

R⁹—CO—NH—(CH₂)₂—N(R¹¹)—CH₂CH₂O—CH₂CH(OH)CH₂—SO₃M² (XI)

R⁹—CO—NH—(CH₂)₂—N(R¹¹)—CH₂CH₂O—CH₂CH(OH)CH₂—OPO₃HM² (XII)

in which R⁹, R¹¹ and M² have the same meanings as in formula (VIII).

Examples of alkylamidoalkylamines are the following compounds: Cocoamphodipropionic Acid. Cocobetainamido Amphopropionate, DEA-Cocam-phodipropionate, Disodium Caproamphodiacetate, Disodium Caproampho-dipropionate, Disodium Capryloamphodiacetate, Disodium Capryloam- phodipropionate, Disodium Cocoamphocarboxyethylhydroxypropylsulfonate, Disodium Cocarnphodiacetate, Disodiur Cocamphodipropionate, Disodiumn Isostearoamphodiacetate, Disodium Isostearoamphodipropionate, Disodium Laureth-5 Carboxyamphodiacetate, Disodium Lauroamphodiacetate, Disodium Lauroamphodipropionate, Disodium Oleoamphodipropionate, Disodium PPG-2-Isodeceth-7 Carboxyamphodiacetate, Disodium Stearoamphodiacetate, Disodium Tallowamphodiacetate, Disodium Vheatgermamphodiacetate, Lauroampho-dipropionic Acid, Quaternium-85, Sodium Caproamphoacetate, Sodium Caproamphohydroxypropylsulfonate, Sodium Caproamphopropionate, Sodium Caprylarphoacetate, Sodium Caprylarnphohydroxypropylsulfonate, Sodiuim Caprylamphopropionate, Sodium Cocoamphoacetate, Sodium Cocoampho-hydroxypropylsulfonate, Sodium Cocoamphopropionate, Sodium Cornapho-propionate, Sodium Isostearoamphoacetate, Sodium Isostearoamphopropionate, Sodium Lauroarnphoacetate, Sodium Lauroamophohydroxypropylsulfonate, Sodium Laurompho PG-Acetate Phosphate, Sodium Lauroamphopropionate, Sodium Myristoamphoacetate, Sodium Oleoamphoacetate, Sodium Oleompho-hydroxypropylsulfonate, Sodium Oleoamphopropionate, Sodium Ricinoleo-amphoacetate, Sodium Stearoamphoacetate, Sodium Stearoampho-hydroxypropylsulfonate, Sodium Stearoamphopropionate, Sodium Tallamphoproprionate, Sodium Tallowamphoacetate, Sodium Undecylenoam-phoacetate, Sodium Undecylenoamphopropionate, Sodium Wheat Germam-phoacetate and Trisodium Lauroampho PG-Acetate Chloride Phosphate.

Alkyl-substituted amino acids

Alkyl-substituted amino acids preferred according to the invention are monoalkyl-substituted amino acids according to formula (XIII)

R¹³—NH—CH(R¹⁴)31 (CH₂)ᵤ—COOM³ (XIII)

in which R ¹³ is a saturated or unsaturated C₆₋₂₂-alkyl radical, preferably C₈₋₁₈-alkyl radical, in particular a saturated C₁₀₋₁₆-alkyl radical, for example a saturated C₁₂₋₁₄-alkyl radical,

R¹⁴ is hydrogen or a C₁₄-alkyl radical, preferably H,

u is a number from 0 to 4, preferably 0 or 1, in particular 1, and

M³ is hydrogen, an alkali metal, an alkaline earth metal or a protonated alkanolamine, e.g. protonated mono-, di- or triethanolamine. alkyl-substituted imino acids according to formula (XIV),

R¹⁵—N—[(CH₂)ᵥ—COOM⁴]₂ (XIV)

in which R¹⁵ is a saturated or unsaturated C₆₋₂₂-alkyl radical, preferably C₈₋₁₈-alkyl radical, in particular a saturated C₁₀₋₁₆-alkyl radical, for example a saturated C ₁₂₋₁₄-alkyl radical,

v is a number from 1 to 5, preferably 2 or 3, in particular 2, and

M⁴ is hydrogen, an alkali metal, an alkaline earth metal or a protonated alkanolamine, e.g. protonated mono-, di- or triethanolamine, where M⁴ in the two carboxyl groups can have the same meanings or two different meanings, e.g. may be hydrogen and sodium or both sodium, and mono- or dialkyl-substituted natural amino acids according to formula (XV).

R¹⁶—N(R¹⁷)—CH(R¹⁸)—COOM⁵ (XV)

in which R¹⁶ is a saturated or unsaturated C₆₋₂₂-alkyl radical, preferably C₈₋₁₈-alkyl radical, in particular a saturated C 0-1₆-alkyl radical, for example a saturated C₁₂₋₁₄-alkyl radical,

R¹⁷ is hydrogen or a C₁₋₄-alkyl radical, optionally hydroxy- or amine-substituted, e.g. a methyl, ethyl, hydroxyethyl or aminopropyl radical,

R¹⁸ is the radical of one of the 20 natural α-amino acids H₂NCH(R²⁰)COOH, and

M⁵ is hydrogen, an alkali metal, an alkaline earth metal or a protonated alkanolamine, e.g. protonated mono-, di- or triethanolamine.

Particularly preferred alkyl-substituted amino acids are the aminopropionates according to formula (XVI),

R¹³—NH—CH₂CH₂COOM³ (XVI)

in which R¹³ and M³ have the same meanings as in formula (XIII).

Examples of alkyl-substituted amino acids are the following compounds: Aminopropyl Laurylglutamine, Cocaminobutyric Acid, DEA-Lauramino-propionate, Disodium Cocarinopropyl Iminodiacetate, Disodium Dicarboxyethyl Cocopropylenediamine, Disodium Lauriminodipropionate, Disodium Stearinminodipropionate, Disodium Tallowiminodipropionate, Lauraminopropionic Acid, Lauryl Aminopropylglycine, Lauryl Diethylenediarinoglycine, Myristaminopropionic Acid, Sodium C₁₂₋₁₅-Alkoxypropyl Iminodipropionate, Sodium Cocarinopropionate, Sodium Lauraminopropionate, Sodium Lauriminodipropionate, Sodium Lauroyl Methylaminopropionate, TEA-Lauraminopropionate and TEA-Myristaminopropionate.

Acylated Amino Acids

Acylated amino acids are amino acids, in particular the 20 natural α-amino acids, which carry, on the amino nitrogen atom, the acyl radical R¹⁹CO of a saturated or unsaturated fatty acid R¹⁹COOH, where R¹⁹ is a saturated or unsaturated C₆₋₂₂-alkyl radical, preferably C₈₋₂₂-alkyl radical, in particular a saturated CI₁O-₆-alkyl radical, for example a saturated C₁₂₋₁₄-alkyl radical. The acylated amino acids can also be used as alkali metal salts, alkaline earth metal salts or alkanolammonium salt, e.g. mono-, di- or triethanolammonium salt. Examples of acylated amino acids are the acyl derivatives, e.g. Sodium Cocoyl Glutamate, Lauroyl Glutamic Acid, Caproyloyl Glycine or Myristoyl Methylanine.

Anionic Surfactants

Anionic surfactants are interface-active compounds with one or more functional anion-active groups which dissociate in aqueous solution to form anions which are ultimately responsible for the interface-active properties.

Suitable anionic surfactants are, for example, fatty alcohol sulfates of fatty alcohols having 8 to 22, preferably 10 to 18, carbon atoms, C₁₂₋₁₈-alcohol sulfates, lauryl sulfate, cetyl sulfate, myristyl sulfate, palmityl sulfate, stearyl sulfate and tallow fatty alcohol sulfate.

Further suitable anionic surfactants are sulfated ethoxylated C₈- to C₂₂-alcohols (alkyl ether sulfates) and soluble salts thereof. Compounds of this type are prepared, for example, by firstly alkoxylating a C₈- to C₂₂-, preferably a C₁₀- to C₁₈-alcohol, e.g. a fatty alcohol, and then sulfating the alkoxylation product. For the alkoxylation, preference is given to using ethylene oxide, with 1 to 50, preferably 1 to 20, mol of ethylene oxide being used per mole of alcohol. The alkoxylation of the alcohols can, however, also be carried out with propylene oxide on its own and if appropriate butylene oxide. Further ore, those alkoxylated C₈- to C₂₂-alcohols which comprise ethylene oxide and propylene oxide or ethylene oxide and butylene oxide or ethylene oxide and propylene oxide and butylene oxide are suitable. The alkoxylated C₈- to C₂₂-alcohols can comprise the ethylene oxide, propylene oxide and butylene oxide units in the form of blocks or in random distribution. Depending on the type of alkoxylation catalyst, it is possible to obtain alkyl ether sulfates with a broad or narrow alkylene oxide homolog distribution.

Further suitable anionic surfactants are alkanesulfonates such as C₈- to C₂₄-, preferably C₁₀- to C₁₈-alkanesulfonates, and soaps, such as, for example, the Na and K salts of saturated and/or unsaturated C₈- to C₂₄-carboxylic acids.

Further suitable anionic surfactants are linear C₈- to C₂₀-alkylbenzenesulfonates (“LAS”), preferably linear C₉- to C₁₃-alkylbenzenesulfonates and -alkyltoluenesulfonates. Analogs thereof with a branched alkyl chain (“BAS”=“branched alkyl sulfonate”) are likewise suitable. The alkyl chain can here result from the reaction of benzene or toluene with, for example, tetramer propylene, trimer butene or dimer hexane.

Further suitable anionic surfactants are C₈- to C₂₄-olefinsulfonates and -disulfonates, which can also represent mixtures of alkene- and hydroxyalkanesulfonates and -disulfonates, respectively, alkyl ester sulfonates, sulfonated polycarboxylic acids, alkyl glycerol sulfonates, fatty acid glycerol ester sulfonates, alkylphenol polyglycol ether sulfonates, paraffin sulfonates having about 20 to about 50 carbon atoms (based on paraffin obtained from natural sources or paraffin mixtures), alkyl phosphates, acyl isethionates, acyl taurates, acylmethyl taurates, alkylsuccinic acids, alkenylsuccinic acids or half-esters or half-amides thereof, alkylsulfosuccinic acids or amides thereof, mono- and diesters of sulfosuccinic acids, acyl sarcosinates, sulfated alkyl polyglucosides, alkyl polyglycol carboxylates and hydroxyalkyl sarcosinates.

The anionic surfactants are preferably used in the form of salts. Suitable cations in these salts are alkali metal ions, such as sodium, potassium and lithium and anunonium salts, such as, for example, hydroxyethylammonium, di(hydroxyethyl)-anmnonium and tri(hydroxyethyl)ammonium salts.

It is possible to use individual anionic surfactants or a combination of different anionic surfactants. It is possible to use anionic surfactants from only one class, for example only fatty alcohol sulfates or only alkylbenzenesulfonates, although it is also possible to use surfactant mixtures from different classes, e.g. a mixture of fatty alcohol sulfates and alkylbenzenesulfonates.

In the method according to the invention, various types of surfactant can be used individually or in a mixture.

The fraction of surfactant or of surfactant mixture in the liquid mist drops, based on the total amount of solvent, is 10 to 3000 ppm by weight, preferably 100 to 1000 ppm by weight, particularly preferably 300 to 1000 ppm by weight.

On account of the low concentration, the surfactants used do not represent a disposal problem, not least because they are often very readily biodegradable.

Solvents which can be used in the method according to the invention are all high-boiling, hydrophilic solvents. Examples are water, high-boiling alcohols, ketones, ethers—such as, for example, alcohol ethoxylate propoxylates with random structure—or mixtures thereof. The solvent preferably comprises ≧85% by weight, particularly preferably ≧90% by weight, very particularly preferably >95% by weight of water or is exclusively water.

The solvent, preferably water, can comprise one or more high-boiling additives, for example ethylene glycol, propylene glycol, ethers, such as, for example, diethylene glycol, dipropylene glycol, poly-THF, polyols, such as, for example, glycerol, polyglycerol, sugars (e.g. glucose, fructose, sucrose, mannose), sugar alcohols (e.g. xylitol, mannitol, sorbitol), trimethylolpropane, pentaerythritol or carboxylic acids, such as, for example, acetic acid, formic acid, oleic acid, benzoic acid, lactic acid.

Based on the total amount of solvent, the amount of additive is less than 15% by weight, preferably less than 10% by weight, particularly preferably less than 5% by weight. If one or more high-boiling additives are present in the solvent, they are present in an amount of at least 0.001% by weight, preferably 0.01% by weight. particularly preferably 1% by weight.

In a further preferred embodiment of the method according to the invention, the liquid mist comprises further additives chosen from the group consisting of
1. cyclodextrins,
2. oxidizing agents, for example H₂O₂ and H₂O₂ storage compounds, such as, for example, peroxodisulfate or peracetic acid, chlorine and chlorine oxide and chlorine-oxygen salts, such as, for example, ClO₂, Cl₂O, NaClOₓ (x=1-4) and the corresponding analogs of fluorine, bromine and iodine, oxidizing transition metal salts, such as, for example, the salts of Mn^{VII} (e.g. KMnO₄), of Ag^{III} (e.g. Ag^{I}[Ag^{III}(SO₄)₂], of Cr_{VI} (e.g. CrO₃ or CrO₂Cl₂), of Pb^{IV} (e.g. Na₂[PbCl₆]) or of Ce^{IV},
3. biocides, for example 2-bromo-2-nitropropane-1,3-diol, phenoxyethanol, phenoxypropanol, aldehydes and aldehyde storage compounds, such as, for example, formaldehyde, glutardialdehyde or hexahydrotriazines, isothiazolinones, such as, for example, methyl-, benzyl- or chloroisothiazolinones,
4. complexing agents for capturing heavy metals, e.g. mercaptans, NTA, EDTA, DTPA, alkali metal sulfides, polycarboxylates,
5. corrosion inhibitors, e.g. wax copolymers, phosphates and phosphate esters, alkanolaminecarboxylic acid salts, amines, boric esters of alkanolamines, polyamines, such as, for example, polyaziridine or polyvinylamine, nitrites,
6. beneficial animals and beneficial microorganisms, such as, for example, Bacillus thuringiensis, bacteriophages, viruses, nematodes,
7. acids or alkalis, such as, for example, H₂SO₄, HCOOH, H₃PO₄, HNO₃, alkanolamines, NaOH, KOH, Ca(OH)₂ and
8. mixtures of 1-7 with or without the addition of surfactants.

Furthermore, the present invention also relates to the use of surfactant-containing suspendable liquid mists for deodorization, decontamination and disinfection, for example in hospitals, doctors surgeries, in the military sector, in nuclear power stations, laboratories, kitchens, canteens, wet rooms, baths and toilets, greenhouses, stables, zoos, smoking areas, apartments, hotel rooms, production plants, interiors of cars, composting works, refuse dumps and heaps, for corrosion protection and for preservation, for example in the metal- and wood-processing industry, in the car trade or in the building trade, for the stripping of solid surfaces, for example of polymer layers or paints, for example in the building industry or in recycling plants or for the placing of beneficial animals and microorganisms onto these, for example in composting works, greenhouses, in farming and forestry or in recycling plants.

1-9. (canceled)

10. A method of deodorizing solid surfaces, wherein a suspendable liquid mist provided with suitable surfactants is applied to the surface to be treated, where the liquid mist drops have a surfactant content of from 10 to 3000 ppm by weight, based on the total amount of solvent, and an average drop size (weight-average) of ≦100 μm, wherein the liquid mist contains oxidizing agents.

11. The method according to claim 10, wherein the solvent comprises at least 85% by weight of water.

12. The method according to claim 10, wherein the surfaces are chosen from the group consisting of polymer surfaces, metallic surfaces, ceramic surfaces, porcelain surfaces, glass surfaces, wood surfaces, paint surfaces, textile surfaces, surfaces of plants, animals or people, leather and hides.

13. The method according to claim 10, wherein the liquid mist comprises further additives chosen from the group consisting of cyclodextrins, biocides, complexing agents for capturing heavy metals, corrosion inhibitors, beneficial animals and useful microorganisms, acids or alkalis and mixtures thereof with or without the addition of surfactants.

14. The method according to claim 10, wherein the surfactants are chosen from cationic, nonionic, zwitterionic, anionic surfactants and mixtures of two or more of said surfactants.

15. The method according to claim 10, wherein a suspendable liquid mist having an average drop size (weight-average) of from 1 to 100 μm in diameter is used.

16. The method according to claim 15, wherein a suspendable liquid mist having an average drop size (weight-average) of from 1 to 50 μm in diameter is used.

17. The method according to claim 10, wherein aqueous liquid mist drops with a surfactant content of from 100 to 1000 ppm by weight, based on the total amount of solvent, are used.
